(19) United States
(12) Patent Application Publication
(10) Pub. No.: US 2023/0030057 A1
(43) Pub. Date: Feb. 2, 2023

(54) PROCESSOR DEVICE AND METHOD OF OPERATING THE SAME

(71) Applicant: FUJIFILM Corporation, Tokyo (JP)

(72) Inventor: Tatsuya AOYAMA, Kanagawa (JP)

(73) Assignee: FUJIFILM Corporation, Tokyo (JP)

(21) Appl. No.: 17/938,617

(22) Filed: Oct. 6, 2022

Related U.S. Application Data
(63) Continuation of application No. PCT/JP2021/007701, filed on Mar. 1, 2021.

(30) Foreign Application Priority Data
Apr. 8, 2020 (JP) 2020-069713

(51) Int. Cl. A61B 1/00; G09G 5/00

(52) U.S. Cl. CPC A61B 1/000094; G09G 5/00; A61B 1/0005; G09G 2380/08; G09G 2320/0247; G09G 2340/12

(57) ABSTRACT

An observation condition acquisition unit acquires an observation condition including at least one of a moving speed of an endoscope, an observation distance between the endoscope and an observation target, or brightness of the observation target. A lesion information acquisition unit acquires lesion information including at least one of a diagnostic purpose or a certainty degree of a lesion obtained from an endoscope image at a timing at which the observation condition is acquired. A display format determination unit determines a display format of the lesion information on a display based on at least any of the observation condition or the lesion information. A control of displaying the lesion information on the display according to the display format is performed.

### CROSS-REFERENCE TO RELATED APPLICATIONS

This application is a Continuation of PCT International Application No. PCT/JP2021/007701 filed on 1 Mar. 2021, which claims priority under 35 U.S.C § 119(a) to Japanese Patent Application No. 2020-069713 filed on 8 Apr. 2020. The above application is hereby expressly incorporated by reference, in its entirety, into the present application.

### BACKGROUND OF THE INVENTION

### 1. Field of the Invention

The present invention relates to a processor device that performs a control of displaying lesion information such as a certainty degree of a lesion on a display and a method of operating the same.

### 2. Description of the Related Art

In a medical field, diagnosis using a medical image has been widely performed. For example, as a device using a medical image, there is an endoscope system comprising a light source device, an endoscope, and a processor device. In the endoscope system, an observation target is irradiated with illumination light, and the observation target illuminated by the illumination light is imaged to acquire an endoscope image as a medical image. The endoscope image is displayed on a monitor and used for diagnosis.

An endoscope system in recent years has been used to support user's diagnosis by detecting and highlighting a region-of-interest, such as a lesion region, from an endoscope image. For example, JP2011-255006A discloses that, in a case where a region-of-interest is detected, whether or not an alert image is to be displayed is determined based on a size of the region-of-interest, and the alert image is displayed or non-displayed based on a result of the determination. Thus, since frequent detection of the region-of-interest is troublesome to a user, the alert image is displayed only in a case where it is necessary to give information to the user, such as a case where a size of a lesion is large.

### SUMMARY OF THE INVENTION

In a case where a lesion is detected or a lesion range is specified based on an endoscope image as disclosed in JP2011-255006A, a non-lesion may be erroneously detected as a lesion or a lesion may be overlooked, depending on an observation condition in a case of acquiring the endoscope image, such as a distance to an observation target, an imaging angle, or brightness of the observation target. Frequent occurrence of the erroneous detection may cause flicker in a case where a result of the detection is superimposed and displayed on an observation image, and may hinder user's diagnosis. Meanwhile, in a case where support such as the detection of the lesion is not used in order to avoid the erroneous detection, the lesion may be overlooked. Therefore, it has been required to appropriately display lesion information according to the observation condition such that lesion is not overlooked while suppressing flicker due to the erroneous detection or the like.

An object of the present invention is to provide a processor device capable of appropriately displaying lesion information according to an observation condition and a method of operating the same.

According to an aspect of the present invention, there is provided a processor device comprising: a processor for image processing, in which the processor for image processing acquires an observation condition including at least one of a moving speed of an endoscope, an observation distance between the endoscope and an observation target, or brightness of the observation target, acquires lesion information including at least one of a diagnostic purpose or a certainty degree of a lesion obtained from an endoscope image at a timing at which the observation condition is acquired, determines a display format of the lesion information on a display based on at least any of the observation condition or the lesion information, and performs a control of displaying the lesion information on the display according to the display format.

It is preferable that the processor for image processing determines the display format by making a display format in a case where the moving speed is a first moving speed and a display format in a case where the moving speed is a second moving speed slower than the first moving speed different from each other. It is preferable that the processor for image processing determines the display format as a display format for non-display in which the lesion information is non-displayed in at least any of a case where the moving speed is the first moving speed or a case where the brightness is less than a brightness threshold value.

It is preferable that the processor for image processing determines the display format as a display format for display in which the lesion information is displayed in a case where the moving speed is the second moving speed and the brightness is equal to or greater than a brightness threshold value. It is preferable that the processor for image processing determines a different display format for display according to the certainty degree in a case where the observation distance is a first observation distance, and determines a different display format for display according to the diagnostic purpose in a case where the observation distance is a second observation distance shorter than the first observation distance.

It is preferable that the processor for image processing determines, as the display format for display, a format in which the lesion information is displayed on the display for each frame in a case where the observation distance is the first observation distance and the certainty degree is equal to or greater than a certainty degree threshold value, and determines, as the display format for display, a first display format for display in which a plurality of specific frames before and after a frame whose certainty degree is less than the certainty degree threshold value are specified and the lesion information is displayed based on first operation processing based on the lesion information of the plurality of specific frames in a case where the observation distance is the first observation distance and the certainty degree is less than the certainty degree threshold value. It is preferable that, in the first display format for display, in a case where there are a specific number or more of frames whose certainty degree is high among the plurality of specific frames, the lesion information is displayed on the display.

It is preferable that the processor for image processing determines, as the display format for display, a second display format for display in which the lesion information related to lesion range diagnosis is displayed based on second operation processing based on the lesion information of a plurality of range diagnosis frames in a case where the observation distance is the second observation distance and the diagnostic purpose is the lesion range diagnosis, and determines, as the display format for display, a third display format for display in which the lesion information related to differential diagnosis is displayed based on third operation processing based on the lesion information of a plurality of differential diagnosis frames in a case where the observation distance is the second observation distance and the diagnostic purpose is the differential diagnosis.

It is preferable that, in the second display format for display, a lesion range is determined based on the lesion information of the plurality of range diagnosis frames, and the lesion information is displayed using the lesion range. It is preferable that, in the third display format for display, a differential content is determined based on the lesion information of the plurality of differential diagnosis frames, and the lesion information is displayed using the differential content. It is preferable that a display image for displaying the lesion information is obtained based on emission of first illumination light, and a lesion information acquisition image for acquiring the lesion information is obtained based on emission of second illumination light having a different emission spectrum from the first illumination light.

According to another aspect of the present invention, provided is a method of operating a processor device including a processor for image processing, in which processor for image processing acquires an observation condition including at least one of a moving speed of an endoscope, an observation distance between the endoscope and an observation target, or brightness of the observation target, acquires lesion information including at least one of a diagnostic purpose or a certainty degree of a lesion obtained from an endoscope image at a timing at which the observation condition is acquired, determines a display format of the lesion information on a display based on at least any of the observation condition or the lesion information, and performs a control of displaying the lesion information on the display according to the display format.

According to the present invention, it is possible to appropriately display the lesion information according to the observation condition.

### BRIEF DESCRIPTION OF THE DRAWINGS

FIG. 1 is an external view of an endoscope system.

FIG. 2 is a block diagram showing a function of the endoscope system.

FIG. 3 is a graph showing spectroscopic transmittance of each color filter of an imaging sensor.

FIG. 4 is a block diagram showing a function of a lesion information processing unit.

FIG. 5 is an image diagram showing a display format for non-display.

FIG. 6 is an image diagram showing a display format for display.

FIG. 7 is an explanatory diagram showing that lesion information is acquired and displayed for each frame.

FIG. 8 is an explanatory diagram showing a first display format for display.

FIG. 9 is an explanatory diagram showing a second display format for display.

FIG. 10 is an explanatory diagram illustrating that a lesion range is reset by second operation processing.

FIG. 11 is an explanatory diagram showing a third display format for display.

FIG. 12 is an image diagram displaying lesion information DIJ using a differential content.

FIG. 13 is a flowchart showing the series of flow of a lesion information display mode.

FIG. 14 is an explanatory diagram showing a first A emission pattern or a second A pattern in an analysis processing mode.

FIG. 15 is an explanatory diagram showing a first B emission pattern in the analysis processing mode.

FIG. 16 is an explanatory diagram showing a second B pattern in the analysis processing mode.

FIG. 17 is an explanatory diagram showing a second C pattern in the analysis processing mode.

FIG. 18 is an explanatory diagram showing a second D pattern in the analysis processing mode.

### DESCRIPTION OF THE PREFERRED EMBODIMENTS

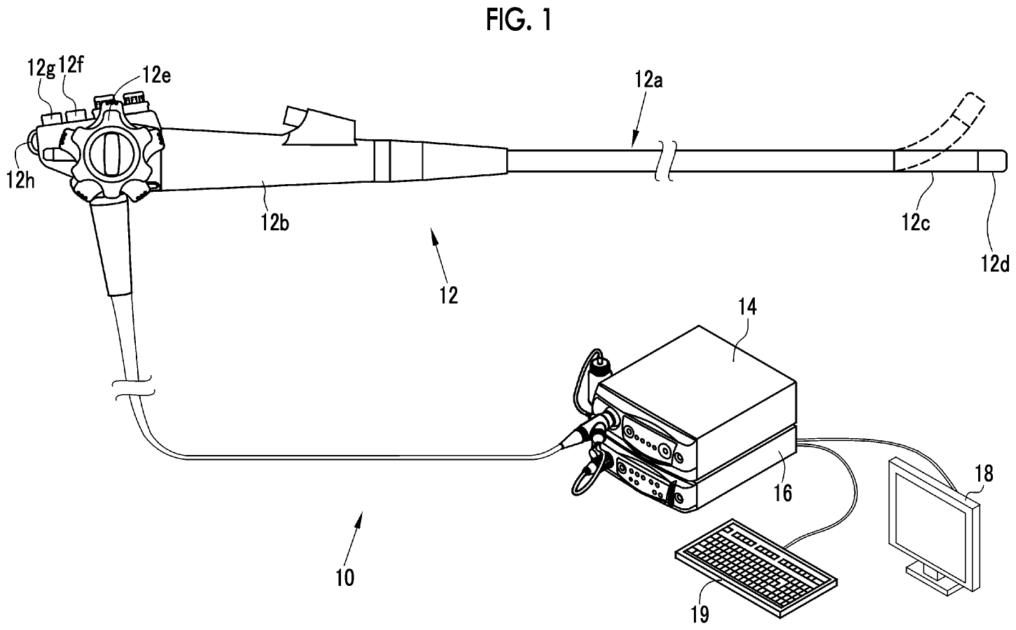

In FIG. 1, an endoscope system 10 includes an endoscope 12, a light source device 14, a processor device 16, a display 18, and a user interface 19. The endoscope 12 is optically connected to the light source device 14 and electrically connected to the processor device 16. The endoscope 12 includes an insertion part 12a to be inserted into a body of an observation target, an operating part 12b provided at a base end portion of the insertion part 12a, and a bendable part 12c and a distal end part 12d provided on a distal end side of the insertion part 12a. The bendable part 12c makes a bending motion by operating an angle knob 12e of the operating part 12b. The distal end part 12d is directed in a desired direction by the bending motion of the bendable part 12c.

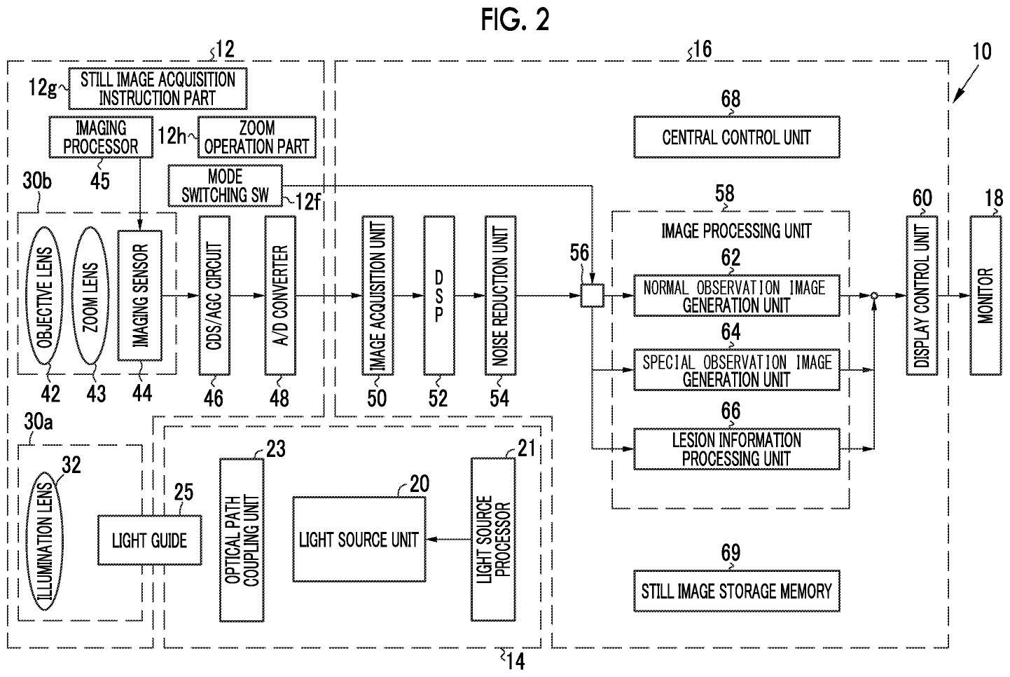

In addition to the angle knob 12e, the operating part 12b is provided with a mode switching SW (mode selector switch) 12f used for a mode switching operation, a still image acquisition instruction part 12g used for providing an instruction of acquisition of a still image of the observation target, and a zoom operation part 12h used for an operation of a zoom lens 43 (see FIG. 2).

The endoscope system 10 has three modes: a normal observation mode, a special observation mode, and a lesion information display mode. In the normal observation mode, the observation target is illuminated with normal light such as white light and an image thereof is captured, so that a normal observation image having a natural color is displayed on the display 18. In the special observation mode, the observation target is illuminated with special light having a wavelength range different from that of normal light and an image thereof is captured, so that a special observation image in which a specific structure is emphasized is displayed on the display 18. In the lesion information display mode, the display format of the lesion information on the display 18 is determined based on at least any of the observation condition or the lesion information, and the lesion information is displayed on the display 18 according to the determined display format. In the lesion information display mode, in addition to continuously emitting either normal light or special light, first illumination light and second illumination light having different emission spectra may be automatically switched to be emitted in a specific emission pattern.

In a case where the user operates the still image acquisition instruction part 12g, a signal related to the still image acquisition instruction is sent to the endoscope 12, the light source device 14, and the processor device 16. In a case where the still image acquisition instruction is given, the still image of the observation target is stored in a still image storage memory 69 (see FIG. 2) of the processor device 16.

The processor device 16 is electrically connected to the display 18 and the user interface 19. The display 18 outputs and displays an image of the observation target, information incidental to the image of the observation target, and the like. The user interface 19 has a keyboard, a mouse, a touch pad, and the like, and has a function of receiving input operations such as function settings. An external recording unit (not shown) for recording an image, image information, or the like may be connected to the processor device 16.

In FIG. 2, the light source device 14 comprises a light source unit 20 and a light source processor 21 that controls the light source unit 20. The light source unit 20 emits illumination light for illuminating the observation target. The light source processor 21 controls the amount of the illumination light emitted from the light source unit 20. The illumination light from the light source unit 20 is incident into a light guide 25 via an optical path coupling unit 23 composed of a mirror, a lens, or the like. The light guide 25 is built in the endoscope 12 and a universal cord (a cord connecting the endoscope 12, the light source device 14 and the processor device 16). The light guide 25 propagates the light from the optical path coupling unit 23 to the distal end part 12d of the endoscope 12.

An illumination optical system 30a and an imaging optical system 30b are provided at the distal end part 12d of the endoscope 12. The illumination optical system 30a has an illumination lens 32, and the illumination light propagated by the light guide 25 is applied to the observation target via the illumination lens 32. The imaging optical system 30b has an objective lens 42 and an imaging sensor 44. The light from the observation target due to the irradiation of the illumination light is incident into the imaging sensor 44 via the objective lens 42 and the zoom lens 43. As a result, an image of the observation target is formed on the imaging sensor 44. The zoom lens 43 is a lens for enlarging the observation target, and moves between a telephoto end and a wide end by operating the zoom operation part 12h.

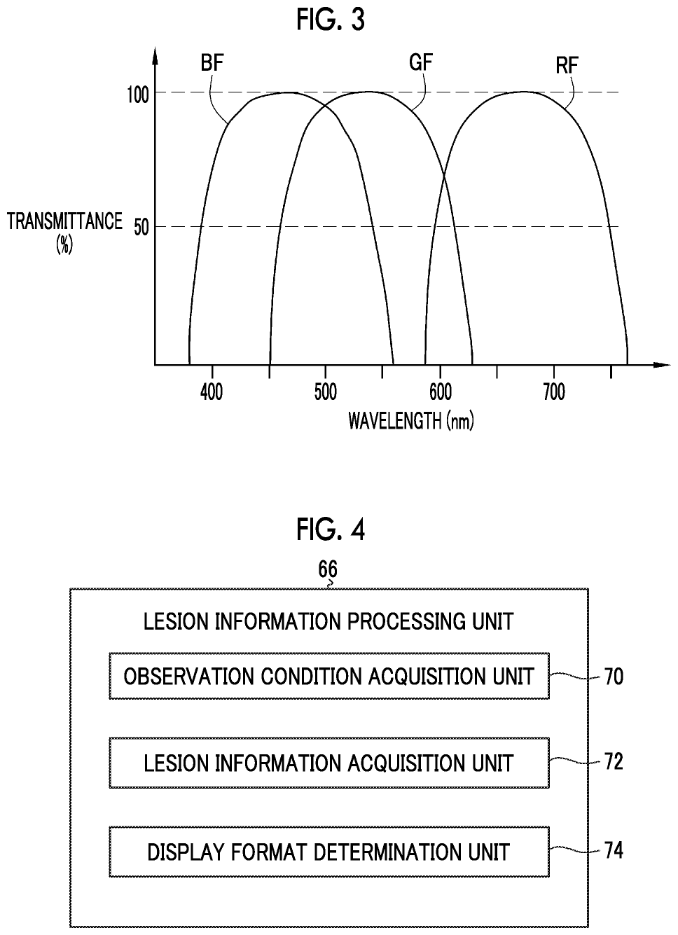

The imaging sensor 44 is a primary color system sensor and comprises three types of pixels: B pixel (blue pixel) having a blue color filter, a G pixel (green pixel) having a green color filter, and an R pixel (red pixel) having a red color filter. As shown in FIG. 3, a blue color filter BF mainly transmits light in a blue band, specifically, light in a wavelength range of 380 to 560 nm. The transmittance of the blue color filter BF peaks in the vicinity of the wavelength of 460 to 470 nm. A green color filter GF mainly transmits light in a green band, specifically, light in a wavelength range of 460 to 620 nm. A red color filter RF mainly transmits light in a red band, specifically, light in a wavelength range of 580 to 760 nm.

The imaging sensor 44 is preferably a charge-coupled device (CCD) or a complementary metal oxide semiconductor (CMOS). The imaging processor 45 controls the imaging sensor 44. Specifically, an image signal is output from the imaging sensor 44 by reading out a signal of the imaging sensor 44 by the imaging processor 45.

As shown in FIG. 2, a correlated double sampling/automatic gain control (CDS/AGC) circuit 46 performs correlated double sampling (CDS) and automatic gain control (AGC) on an analog image signal obtained from the imaging sensor 44. The image signal that has passed through the CDS/AGC circuit 46 is converted into a digital image signal by an analog/digital (A/D) converter 48. The digital image signal after A/D conversion is input to the processor device 16.

The processor device 16 comprises an image acquisition unit 50, a digital signal processor (DSP) 52, a noise reduction unit 54, an image processing switching unit 56, an image processing unit 58, and a display control unit 60. The image processing unit 58 comprises a normal observation image generation unit 62, a special observation image generation unit 64, and a lesion information processing unit 66.

In the processor device 16, a program for performing various processes such as a process related to lesion information is stored in a program memory (not shown). In a case where a central control unit 68 composed of a processor for image processing executes the program in the program memory, functions of the image acquisition unit 50, the digital signal processor (DSP) 52, the noise reduction unit 54, the image processing switching unit 56, the image processing unit 58, and the display control unit 60 are realized. Along with this, functions of the normal observation image generation unit 62, the special observation image generation unit 64, and the lesion information processing unit 66 included in the image processing unit 58 are realized. The lesion information processing unit 66 realizes functions of an observation condition acquisition unit 70, a lesion information acquisition unit 72, and a display format determination unit 74 (see FIG. 4).

The image acquisition unit 50 acquires an endoscope image input from the endoscope 12. The endoscope image is preferably a color image composed of a blue signal (B image signal), a green signal (G image signal), and a red signal (R image signal) output from the B pixel, G pixel, and R pixel of the imaging sensor 44. The acquired color image is transmitted to the DSP 52. The DSP 52 performs, on the received color image, various signal processes such as a defect correction process, an offset process, a gain correction process, a matrix process, a gamma conversion process, a demosaicing process, and an YC conversion process. In the defect correction process, a signal of a defective pixel of the imaging sensor 44 is corrected. In the offset process, a dark current component is removed from the image signal that has passed through the defect correction process, and an accurate zero level is set. The gain correction process adjusts a signal level of the color image by multiplying the image signal of each color after the offset process by a specific gain coefficient. In a case where a monochrome sensor is used as the imaging sensor 44, the endoscope image is preferably a monochrome image of a plurality of colors output from the monochrome sensor after being imaged by the monochrome sensor for each light emission of a specific color.

The image signal of each color after the gain correction process is subjected to the matrix process for enhancing color reproducibility. After that, the brightness and chroma saturation of the color image are adjusted by the gamma conversion process. The color image after the matrix process is subjected to the demosaicing process (also referred to as an isotropic process), and a signal of a color missing from each pixel is generated by interpolation. By the demosaicing process, all pixels have signals of respective colors of RGB. The DSP 52 performs the YC conversion process on the color image after the demosaicing process, and outputs a brightness signal Y, a color difference signal Cb, and a color difference signal Cr to the noise reduction unit 54.

The noise reduction unit 54 performs a noise reduction process by, for example, a moving average method or a median filter method on the color image that has passed through the demosaicing process or the like by the DSP 52. The color image with reduced noise is input to the image processing switching unit 56.

The image processing switching unit 56 switches a transmission destination of the image signal from the noise reduction unit 54 to any of the normal observation image generation unit 62, the special observation image generation unit 64, or the lesion information processing unit 66 according to the set mode. Specifically, in a case where the normal observation mode is set, the image signal from the noise reduction unit 54 is input to the normal observation image generation unit 62. In a case where the special observation mode is set, the image signal from the noise reduction unit 54 is input to the special observation image generation unit 64. In a case where the lesion information display mode is set, the image signal from the noise reduction unit 54 is input to the lesion information processing unit 66.

The normal observation image generation unit 62 performs image processing for a normal observation image on the input endoscope image. The image processing for a normal observation image includes a color conversion process such as a 3×3 matrix process, a gradation transformation process, and a three-dimensional look up table (LUT) process, a color enhancement process, and a structure enhancement process such as a spatial frequency enhancement. The endoscope image that has passed through the image processing for a normal observation image is input to the display control unit 60 as a normal observation image.

The special observation image generation unit 64 performs image processing for a special observation image on the input endoscope image. The image processing for a special observation image includes a color conversion process such as a 3×3 matrix process, a gradation transformation process, and a three-dimensional look up table (LUT) process, a color enhancement process, and a structure enhancement process such as a spatial frequency enhancement. The endoscope image that has passed through the image processing for a special observation image is input to the display control unit 60 as a special observation image.

The lesion information processing unit 66 acquires the observation condition, extracts the lesion information, and determines the display format of the lesion information based on the input endoscope image. The endoscope image, the lesion information, and the display format of the lesion information are transmitted to the display control unit 60. The details of the lesion information processing unit 66 will be described below.

The display control unit 60 performs a control of displaying the image or the like output from the image processing unit 58 on the display 18. Specifically, in a case of the normal observation mode or the special observation mode, the display control unit 60 converts the normal observation image or the special observation image into a video signal that enables full-color display on the display 18. The converted video signal is input to the display 18. As a result, the normal observation image or the special observation image is displayed on the display 18.

In a case of the lesion information display mode, the display control unit 60 enables the endoscope image to be displayed in full color on the display 18 and converts the lesion information according to the display format of the lesion information into a video signal that enables display on the display 18. The converted video signal is input to the display 18. As a result, the display 18 displays an endoscope image on which the lesion information is superimposed and displayed.

As shown in FIG. 4, the lesion information processing unit 66 comprises the observation condition acquisition unit 70, the lesion information acquisition unit 72, and the display format determination unit 74. The observation condition acquisition unit 70 acquires observation conditions including at least one of a moving speed of the endoscope 12, an observation distance between the endoscope 12 and the observation target, or brightness of the observation target. The observation condition refers to other conditions including an imaging condition at a timing at which the observation target is imaged by the user.

Specifically, the observation conditions include a moving speed of the distal end part 12d of the endoscope 12. The moving speed is acquired based on a difference comparison with an endoscope image of several frames before or after a frame obtained at a timing at which imaging is performed (simple block matching with limited sub-block and limited search range) and motion information of the distal end part 12d obtained from a position information sensor (not shown) provided at the distal end part 12d of the endoscope 12. The moving speed is used to determine whether a timing at which the user is performing endoscopic observation is a timing at which the lesion is being detected or a timing at which the distal end part 12d is simply moving to a target site. The frame is a unit of a period including at least a period from a specific timing to the completion of signal reading in the imaging sensor 44.

The observation distance is preferably expressed by, for example, a distance between the distal end part 12d of the endoscope 12 and the observation target. As the observation distance, a zoom level at which the observation target is enlarged or reduced by operating the zoom operation part 12h may be used. For example, the zoom level is determined by a magnification ratio of the observation target (no magnification, 25×, 50×, 75×, 125×, or the like). In addition, as the observation distance, distance information obtained based on irradiation position of a laser beam for distance measurement from the distal end part 12d of the endoscope 12 on the observation target after irradiating the observation target with the laser beam for distance measurement may be used. In addition, as the observation distance, distance information obtained from an area of a halation region (region where a brightness value is extremely high) generated by illumination light emitted from the distal end part 12d of the endoscope 12 may be used. In this case, in a case where the area of the halation region is large, the observation distance is short, and in a case where the area of the halation region is small, the observation distance is long. The observation distance is used to determine whether the timing at which the user is performing endoscopic observation is a timing at which presence diagnosis for detecting the presence of the lesion is being performed, a timing at which lesion range diagnosis for determining a range of the lesion is being performed, or a timing at which differential diagnosis for differentiating the lesion, such as a stage of the lesion, is being performed.

The brightness of the observation target is preferably calculated based on the endoscope image. For example, the brightness of the observation target may be an average value of the entire pixel values of the endoscope image, and may be a value obtained based on an area of a dark region whose pixel value is equal to or less than a specific value among effective pixel regions of the endoscope image. The brightness of the observation target is used to determine whether or not the brightness is suitable for detecting the lesion or the like at a timing at which the user is performing endoscopic observation.

The lesion information acquisition unit 72 acquires lesion information at least including a diagnostic purpose or a certainty degree of the lesion obtained from the endoscope image at a timing at which the observation condition is acquired. The certainty degree of the lesion is preferably calculated by performing an artificial intelligence (AI) process on the endoscope image. The certainty degree of the lesion is preferably expressed by a numerical value such as “60” or “80”. As the AI process, it is preferable to use a convolutional neural network (CNN). The diagnostic purpose is preferably input by the user via the user interface 19. The diagnostic purpose includes presence diagnosis for detecting the presence of the lesion, lesion range diagnosis for determining a range of the lesion, differential diagnosis for differentiating the lesion, such as a stage of the lesion, or the like. As the lesion information, information obtained based on characteristics such as blood vessel density and density distribution, blood vessel thickness variation and variation distribution, and blood vessel diameter distribution and bleeding presence or absence, and regularity and complexity of blood vessels and surface structures by AI by extracting blood vessel information from the endoscope image may be used.

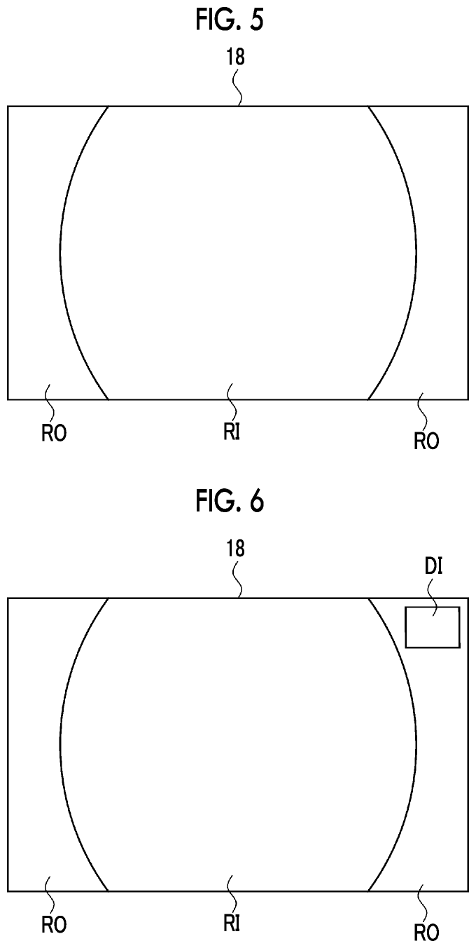

The display format determination unit 74 determines a display format of the lesion information on the display 18 based on at least any of the observation condition or the lesion information. As the display format of the lesion information, as shown in FIG. 5, there is a display format for non-display in which the lesion information is non-displayed in either an inside-of-observation image display region RI for displaying the endoscope image or an outside-of-observation image display region RO for displaying information other than the endoscope image outside the observation image display region. In addition, as the display format of the lesion information, as shown in FIG. 6, there is a display format for display in which lesion information DI is displayed in at least any of an inside-of-observation image display region RI for displaying the endoscope image or an outside-of-observation image display region RO for displaying information other than the endoscope image outside the observation image display region.

The details of a determination method of the display format by the display format determination unit 74 will be described below. The display format determination unit 74 determines the display format by making a display format in a case where the moving speed is a first moving speed and a display format in a case where the moving speed is a second moving speed slower than the first moving speed different from each other. The first moving speed is a high speed at which a speed threshold value exceeds a certain value, and it is considered that a situation where the distal end part 12d of the endoscope is moved at the first moving speed is a situation where the distal end part 12d is moved to an observation site, and the purpose of the movement is not to acquire the lesion information. Therefore, in a case where the moving speed is the first moving speed, the display format determination unit 74 determines the display format of the lesion information as the display format for non-display. In a case of a dark situation where the brightness of the observation target is less than the brightness threshold value, it is considered that the detection of the lesion information is unreliable. Therefore, the display format determination unit 74 determines the display format of the lesion information as the display format for non-display even in a case where the brightness of the observation target is less than the brightness threshold value.

The display format determination unit 74 determines the display format of the lesion information as the display format for display in a case where the moving speed is the second moving speed and the brightness of the observation target is equal to or greater than the brightness threshold value. The second moving speed is a slow speed at which the speed threshold value is less than a certain value, and it is considered that a situation where the distal end part 12d of the endoscope is moved at the second moving speed is intended to acquire the lesion information. In a situation where the lesion information is acquired, a type of the lesion information to be acquired is different depending on the observation distance in many cases, so that it is preferable to use a different display format for display depending on the observation distance.

Specifically, the display format determination unit 74 determines a different display format for display according to the certainty degree of the lesion in a case where the observation distance is a first observation distance, and determines a different display format for display according to the diagnostic purpose in a case where the observation distance is a second observation distance shorter than the first observation distance. The first observation distance is preferably a distance of distant view observation performed in a situation such as screening. The second observation distance is preferably a distance of close view observation performed in a situation such as the lesion range diagnosis or the differential diagnosis.

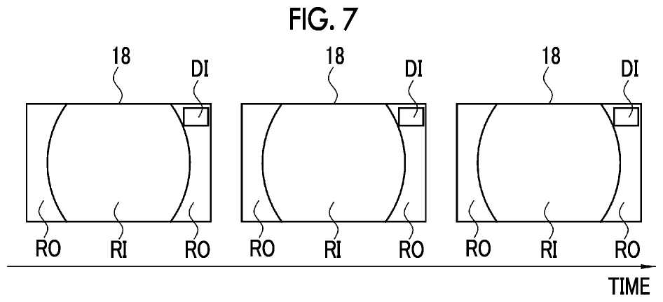

In a case where the observation distance is the first observation distance and the certainty degree of the lesion is equal to or greater than a certainty degree threshold value, the display format determination unit 74 preferably determines, as the display format for display, a format in which the lesion information is displayed on the display 18 for each frame. In this case, for example, as shown in FIG. 7, the certainty degree of the lesion, which is one of the lesion information DIs, is continuously displayed for each frame. In FIG. 7, instead of or in addition to displaying the certainty degree of the lesion as a numerical value, the certainty degree may be displayed as a graph in the outside-of-observation image display region RO. The lesion information may be displayed in the inside-of-observation image display region RI. For example, the lesion information may be visualized and overlay-displayed on the observation image according to the user's instruction.

On the other hand, in a case where the certainty degree of the lesion is less than the certainty degree threshold value in the first observation distance, as the display format for display, a first display format for display is preferably determined in which a plurality of specific frames before and after a frame whose certainty degree is less than the certainty degree threshold value are specified and the lesion information is displayed on the display 18 based on first operation processing based on the lesion information of the plurality of specific frames. Specifically, it is preferable that, in the first display format for display, in a case where there are a specific number or more of the frames whose certainty degree is equal to or greater than a certain value among the plurality of frames, the lesion information is displayed on the display 18. This is to prevent the display of the lesion information from not being displayed in order to avoid overlooking the lesion while suppressing flicker caused by the continuous display of the lesion information in a case where the certainty degree of the lesion is less than the certainty degree threshold value.

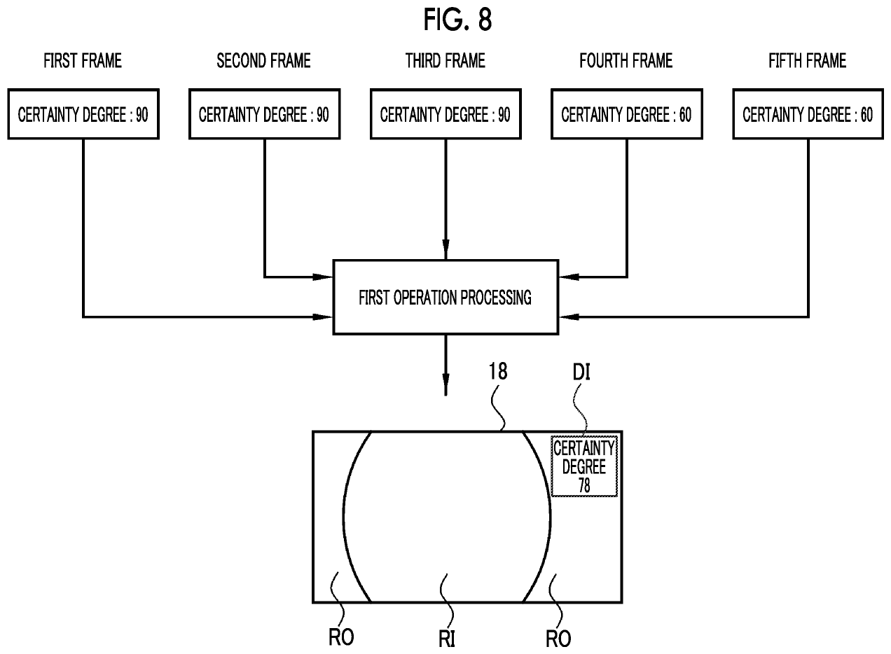

For example, as shown in FIG. 8, in a case where the certainty degree of the lesion in the fifth frame is “60” which is equal to or less than a certain value (for example, “80”), the fifth frame and the first to fourth frames before the fifth frame are specified as a plurality of specific frames. In a case where the specific number, which is a criterion for determining whether to display the lesion information, is set to three frames, since the certainty degree of the first to third frames out of the first to fifth frames is equal to or greater than a certain value of “80”, the number of the frames whose certainty degree is equal to or greater than the certain value is equal to or greater than a specific number of “three frames”. In this case, in the fifth frame, the lesion information is displayed on the display 18 based on the first operation processing based on the lesion information in the first to fifth frames.

As the display content of the lesion information, it is preferable that the content is obtained by performing, for example, a process of calculating a representative value (average value, maximum value) of the certainty degree of the first to fifth frames as the first operation processing. In FIG. 8, “78”, which is an average value of the certainty degree of the first to fifth frames, is displayed in the outside-of-observation image region RO as the lesion information DI. The lesion information may be displayed as a graph in addition to the numerical information. The lesion information may be displayed in the inside-of-observation image region RI. For example, the lesion information may be visualized and overlay-displayed on the observation image according to the user's instruction.

In a case where the observation distance is the second observation distance and the diagnostic purpose is the lesion range diagnosis, the display format determination unit 74 determines, as the display format for display, a second display format for display in which the lesion information related to the lesion range diagnosis is displayed on the display 18 based on second operation processing based on lesion information of a plurality of range diagnosis frames. It is preferable that, in the second display format for display, a lesion range is determined based on the lesion information of the plurality of range diagnosis frames, and the lesion information is displayed on the display 18 using the lesion range.

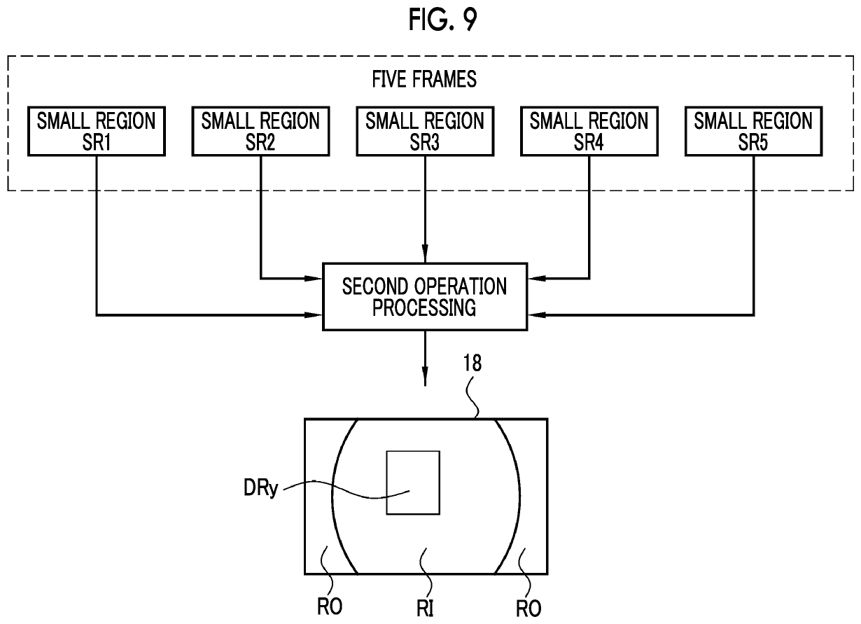

In a case where the diagnostic purpose is set to the lesion range diagnosis, the lesion information acquisition unit 72 calculates the certainty degree of the lesion for each pixel or small region of the endoscope image, and integrates the pixels and small regions whose certainty degree is equal to or greater than a range threshold value, to set a lesion range DRx. In a case where the plurality of range diagnosis frames are defined as five frames and the lesion information related to the lesion range is to be displayed as the lesion information, as shown in FIG. 9, the display format determination unit 74 calculates an average value of the certainty degree of small regions SR1 to SR5 for five frames as the second operation processing, and integrates the small regions whose average value is equal to or greater than a range threshold value, to obtain a resetting lesion range.

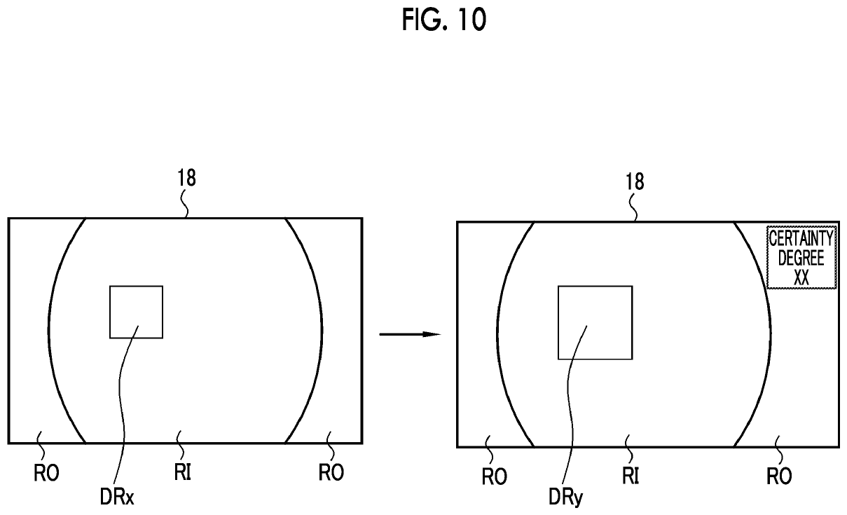

As shown in FIG. 10, as the second operation processing, the lesion range DRx before resetting is reset to a resetting lesion range DRy. Then, overlay-display is performed in the inside-of-observation image region RI such that a portion corresponding to the resetting lesion range is emphasized. In addition, it is preferable to display a representative value (such as average value (displayed as certainty degree XX in FIG. 10)) of the certainty degree in the lesion range DRy in the outside-of-observation image region RO. As a result, variation of the lesion range for each frame is suppressed, so that flicker can be reduced. The small region is preferably a region having a plurality of pixels in a vertical direction. The certainty degree may not be displayed in the outside-of-observation image region RO. In addition, it is preferable to display the lesion information using the lesion range in a cycle of a plurality of range diagnosis frames.

In a case where the observation distance is the second observation distance and the diagnostic purpose is the differential diagnosis, the display format determination unit 74 determines, as the display format for display, a third display format for display in which the lesion information related to the differential diagnosis is displayed on the display 18 based on third operation processing based on lesion information of a plurality of differential diagnosis frames. In the third display format for display, a differential content is determined based on the lesion information of the plurality of differential diagnosis frames, and the lesion information is displayed on the display 18 using the differential content.

In a case where the diagnostic purpose is set to the differential diagnosis, the lesion information acquisition unit 72 integrates characteristics of each pixel and small region of the endoscope image to determine the severity, stage, and certainty degree of a lesion region for each frame. For example, as the stage and certainty degree of the lesion region, in a case of Barrett's esophagus, there are stages of “Barrett without dysplasia”, “high grade dysplasia”, and “adenocarcinoma”, and the certainty degree is expressed as “adenocarcinoma: 60”. In a case of colorectal cancer, there are stages of “benign polyp”, “adenoma”, and “adenocarcinoma”, and the certainty degree is expressed as “benign polyp: 80”.

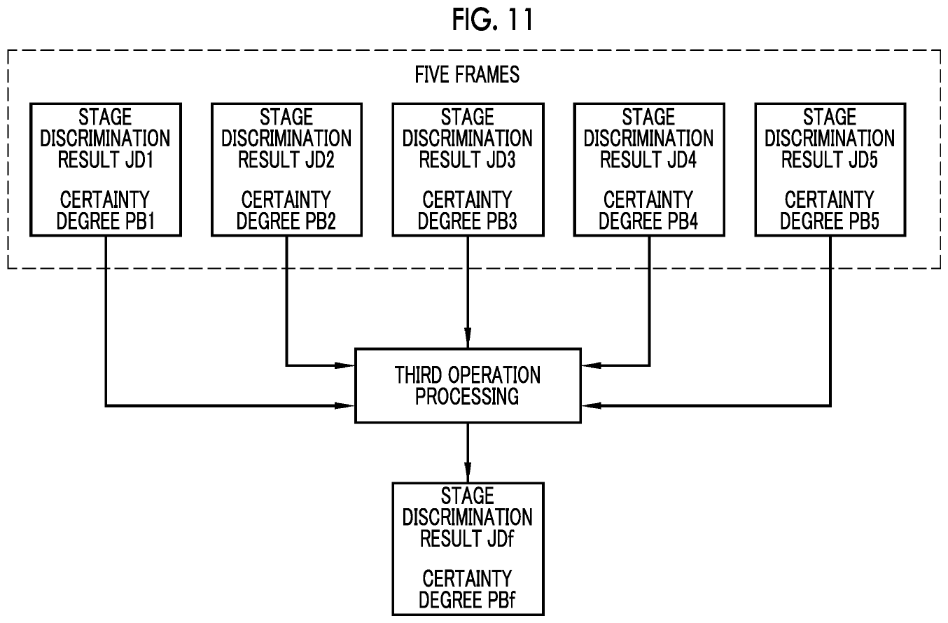

In a case where the plurality of differential diagnosis frames are defined as five frames, as shown in FIG. 11, the display format determination unit 74 calculates a final stage discrimination result JDf and a final certainty degree PBf based on stage discrimination results JD1 to JD5 and certainty degrees PB1 to PB5 for five frames as the third operation processing, and displays the final stage discrimination result JDf and the final certainty degree PBf on the display 18 as the lesion information using the differential content.

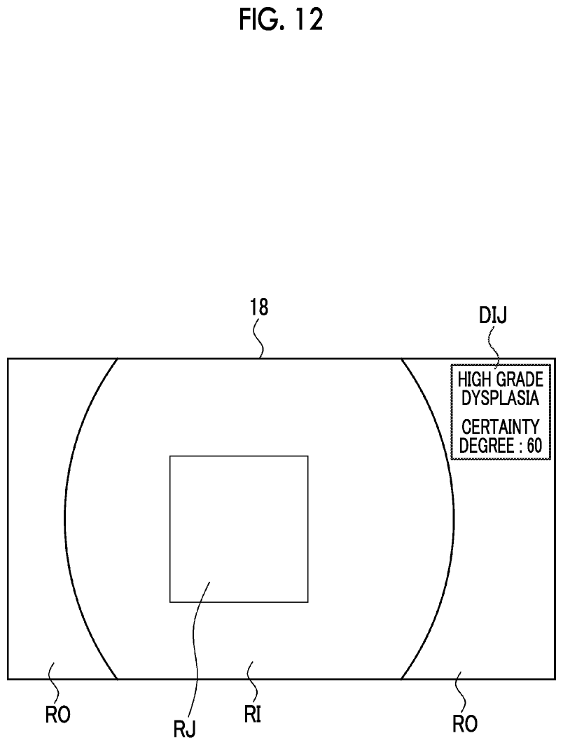

For example, in a case where the differential diagnosis is Barrett's esophageal differentiation and four frames out of the stage discrimination results for five frames are “high grade dysplasia”, “high grade dysplasia” is defined as the final stage discrimination result JDf. A representative value (such as average value) “60” of the certainty degree of the four frames in which the stage is discriminated as “high grade dysplasia” is defined as the final certainty degree PBf. Then, as shown in FIG. 12, as display of lesion information DIJ using the differential content, a region RJ included in a specific range of the final certainty degree “60” is highlighted in the inside-of-observation image region RI, and “high grade dysplasia, certainty degree: 60” is displayed in the outside-of-observation image region RO. The certainty degree may be displayed as a graph. The certainty degree may not be displayed in the outside-of-observation image region RO. In addition, it is preferable to display the lesion information using the differential content in a cycle of a plurality of range diagnosis frames.

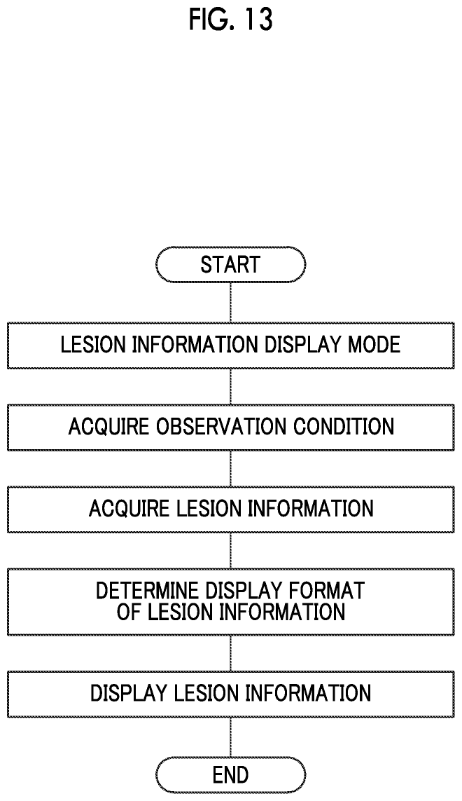

Next, a series of flow of the lesion information display mode will be described with reference to the flowchart of FIG. 13. In a case where the user performs mode switch to the lesion information display mode by operating the mode selector switch 12f, acquisition of the observation condition is started, and acquisition of the lesion information is started at a timing at which the observation condition is acquired. The observation information includes at least one of the moving speed of the endoscope 12, the observation distance between the endoscope 12 and the observation target, or the brightness of the observation target. The lesion information includes at least one of the certainty degree of the lesion obtained from the endoscope image, or the diagnostic purpose.

In a case where the acquisition of the observation condition and the acquisition of the lesion information are completed, the display format determination unit 74 determines the display format of the lesion information on the display 18 based on at least any of the observation condition or the lesion information. The display control unit 60 displays the lesion information on the display 18 according to the display format determined by the display format determination unit 74.

In the lesion information display mode, in a case where first illumination light and second illumination light having different emission spectra are automatically switched to be emitted, the first illumination light is emitted in a first emission pattern and the second illumination light is emitted in a second emission pattern. By switching and emitting the first illumination light and the second illumination light in frame units, a display image for displaying the lesion information can be acquired based on the emission of the first illumination light, and a lesion information acquisition image for acquiring the lesion information can be acquired based on the emission of the second illumination light.

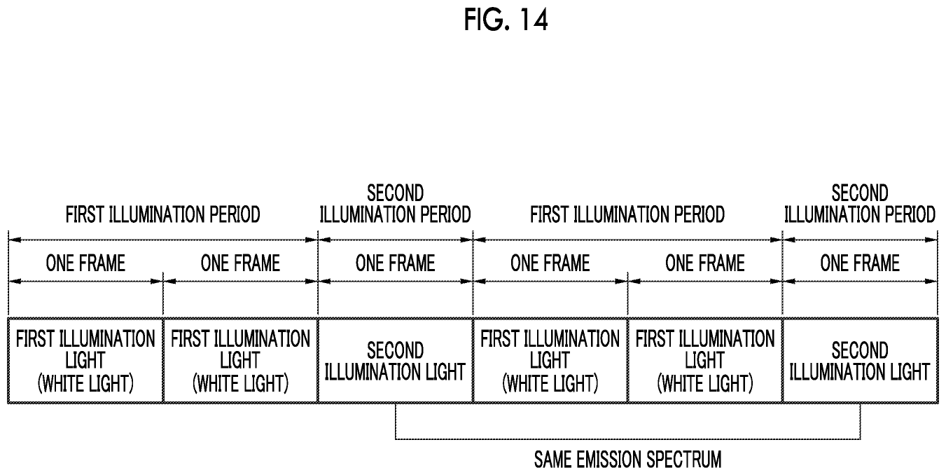

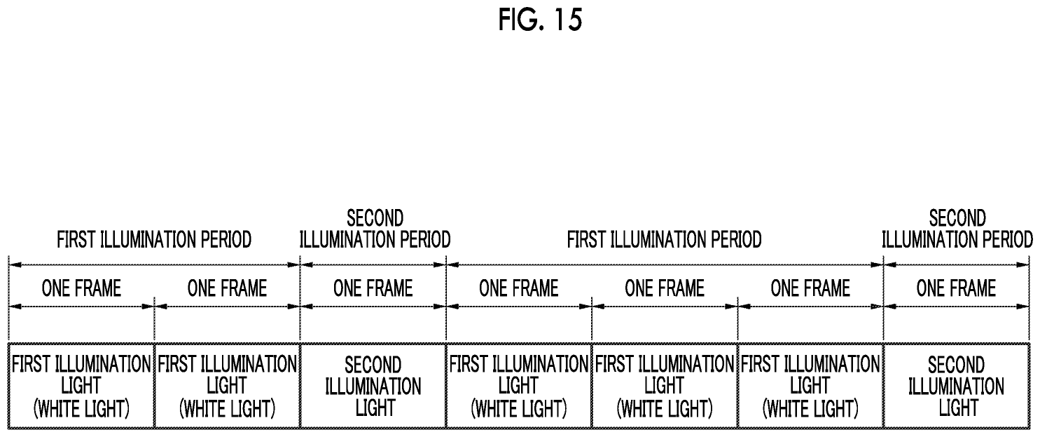

Specifically, the first emission pattern is preferably any of a first A emission pattern in which the number of frames in a first illumination period for emitting the first illumination light is the same in the respective first illumination periods as shown in FIG. 14, or a first B emission pattern in which the number of frames in a first illumination period is different in the respective first illumination periods as shown in FIG. 15. In FIGS. 14 and 15, a second illumination period indicates a period during which the second illumination light is emitted. The period is represented by the number of frames.

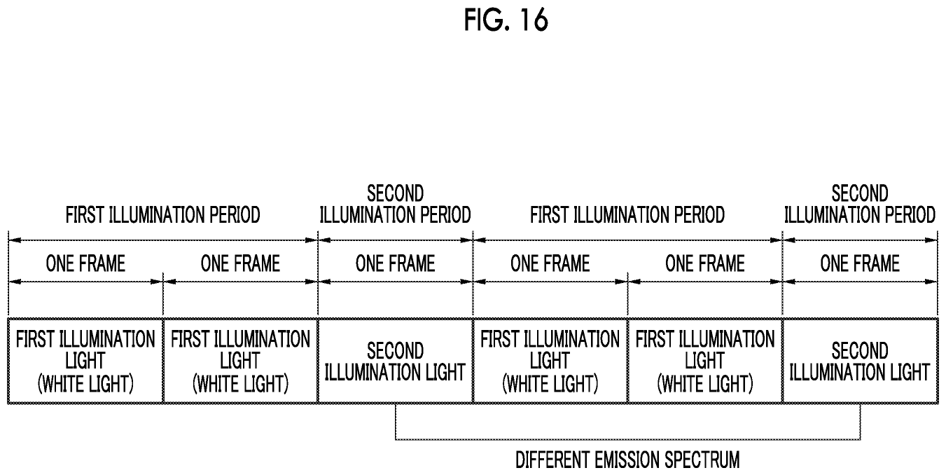

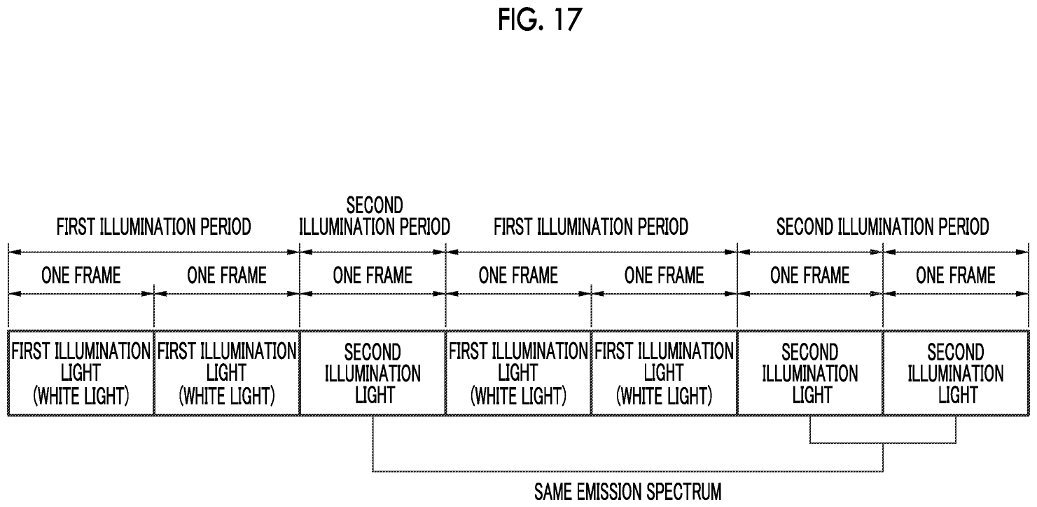

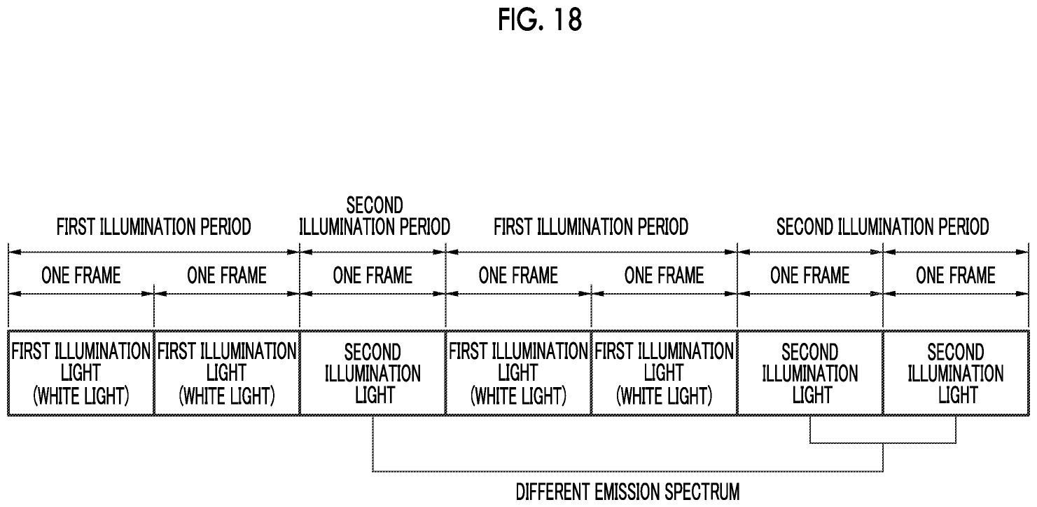

The second emission pattern is preferably any of a second A pattern in which the number of frames in the second illumination period is the same in the respective second illumination periods and the emission spectrum of the second illumination light is the same in the respective second illumination periods as shown in FIG. 14, a second B pattern in which the number of frames in the second illumination period is the same in the respective second illumination periods and the emission spectrum of the second illumination light is different in the respective second illumination periods as shown in FIG. 16, a second C pattern in which the number of frames in the second illumination period is different in the respective second illumination periods and the emission spectrum of the second illumination light is the same in the respective second illumination periods as shown in FIG. 17, or a second D pattern in which the number of frames in the second illumination period is different in the respective second illumination periods and the emission spectrum of the second illumination light is different in the respective second illumination periods as shown in FIG. 18. The emission spectrum of the first illumination light may be the same or different in the respective first illumination periods.

Here, the first illumination period is preferably longer than the second illumination period, and the first illumination period is preferably two frames or more. For example, in FIG. 14, in a case where the first emission pattern is the first A pattern and the second emission pattern is the second A pattern (the number of frames in the second illumination period: the same, the emission spectrum of the second illumination light: the same), the first illumination period is set to two frames and the second illumination period is set to one frame. Since the first illumination light is used for generating a display image to be displayed on the display 18, it is preferable to obtain a bright image by illuminating the observation target with the first illumination light.

For example, the first illumination light is preferably white light. On the other hand, since the second illumination light is used for acquiring the lesion information, it is preferable to obtain an image suitable for acquiring the lesion information by illuminating the observation target with the second illumination light. For example, the second illumination light is preferably short-wavelength narrow-band light such as purple light.

In the above embodiment, although the display format of the lesion information is determined in real time based on the observation condition or the lesion information, the display format of the lesion information may be determined in advance for each observation condition or lesion information in consideration of the real time property, and the display format corresponding to the acquired observation condition or lesion information may be selected from the determined display formats.

In the above embodiment, hardware structures of processing units that execute various kinds of processing, such as the light source processor 21, the imaging processor 45, the image acquisition unit 50, the DSP 52, the noise reduction unit 54, the image processing switching unit 56, the normal observation image generation unit 62, the special observation image generation unit 64, and the lesion information processing unit 66, which are included in the image processing unit 58, the central control unit 68, the observation condition acquisition unit 70, the lesion information acquisition unit 72, and the display format determination unit 74, are various processors as shown below. The various processors include a central processing unit (CPU) that is a general-purpose processor that executes software (programs) to function as various processing units, a programmable logic device (PLD) that is a processor capable of changing a circuit configuration after manufacture, such as a field programmable gate array (FPGA), and an exclusive electric circuit that is a processor having a circuit configuration exclusively designed to execute various kinds of processing.

One processing unit may be constituted by one of these various processors, or may be constituted by a combination of two or more processors of the same type or different types (for example, a combination of a plurality of FPGAs or a combination of a CPU and an FPGA). A plurality of processing units may be constituted by one processor. As an example in which the plurality of processing units are constituted by one processor, first, as represented by a computer such as a client or a server, one processor is constituted by a combination of one or more CPUs and software and this processor functions as the plurality of processing units. Second, as represented by a system on chip (SoC) or the like, a processor that realizes the functions of the entire system including the plurality of processing units by using one integrated circuit (IC) chip is used. As described above, the various processing units are constituted by using one or more of the above described various processors as a hardware structure.

Further, the hardware structure of these various processors is more specifically an electric circuit (circuitry) in a form in which circuit elements such as semiconductor elements are combined. In addition, the hardware structure of the storage unit is a storage device such as a hard disc drive (HDD) or a solid state drive (SSD).

### EXPLANATION OF REFERENCES

10: endoscope system
12: endoscope
12a: insertion part
12b: operating part
12c: bendable part
12d: distal end part
12e: angle knob
12f: mode selector switch
12g: still image acquisition instruction part
12h: zoom operation part
14: light source device
16: processor device
18: display
19: user interface
20: light source unit
21: light source processor
23: optical path coupling unit
25: light guide
30a: illumination optical system
30b: imaging optical system
32: illumination lens
42: objective lens
43: zoom lens
44: imaging sensor
45: imaging processor
46: CDS/AGC circuit
48: A/D converter
50: image acquisition unit
52: DSP
54: noise reduction unit
56: image processing switching unit
58: image processing unit
60: display control unit
62: normal observation image generation unit
64: special observation image generation unit
66: lesion information processing unit
68: central control unit
69: still image storage memory
70: observation condition acquisition unit
72: lesion information acquisition unit
74: display format determination unit

What is claimed is:

1. A processor device comprising:
a processor, configured to:
acquire an observation condition including at least one of a moving speed of an endoscope, an observation distance between the endoscope and an observation target, or brightness of the observation target;
acquire lesion information including at least one of a diagnostic purpose or a certainty degree of a lesion obtained from an endoscope image at a timing at which the observation condition is acquired;
determine a display format of the lesion information on a display based on at least any of the observation condition or the lesion information; and
perform a control of displaying the lesion information on the display according to the display format.

2. The processor device according to claim 1,
wherein the processor is further configured to determine the display format by making a display format in a case where the moving speed is a first moving speed and a display format in a case where the moving speed is a second moving speed slower than the first moving speed different from each other.

3. The processor device according to claim 2,
wherein the processor is further configured to determine the display format as a display format for non-display in which the lesion information is non-displayed in at least any of a case where the moving speed is the first moving speed or a case where the brightness is less than a brightness threshold value.

4. The processor device according to claim 2,
wherein the processor is further configured to determine the display format as a display format for display in which the lesion information is displayed in a case where the moving speed is the second moving speed and the brightness is equal to or greater than a brightness threshold value.

5. The processor device according to claim 4,
wherein the processor is further configured to:
determine a different display format for display according to the certainty degree in a case where the observation distance is a first observation distance; and
determine a different display format for display according to the diagnostic purpose in a case where the observation distance is a second observation distance shorter than the first observation distance.

6. The processor device according to claim 5,
wherein the processor is further configured to:
determine, as the display format for display, a format in which the lesion information is displayed on the display for each frame in a case where the observation distance is the first observation distance and the certainty degree is equal to or greater than a certainty degree threshold value; and
determine, as the display format for display, a first display format for display in which a plurality of specific frames before and after a frame whose certainty degree is less than the certainty degree threshold value are specified and the lesion information is displayed based on first operation processing based on the lesion information of the plurality of specific frames in a case where the observation distance is the first observation distance and the certainty degree is less than the certainty degree threshold value.

7. The processor device according to claim 6,
wherein, in the first display format for display, the lesion information is displayed on the display in a case where there are a specific number or more of frames whose certainty degree is high among the plurality of specific frames.

8. The processor device according to claim 5,
wherein the processor is further configured to:
determine, as the display format for display, a second display format for display in which the lesion information related to lesion range diagnosis is displayed based on second operation processing based on the lesion information of a plurality of range diagnosis frames in a case where the observation distance is the second observation distance and the diagnostic purpose is the lesion range diagnosis, and
determine, as the display format for display, a third display format for display in which the lesion information related to differential diagnosis is displayed based on third operation processing based on the lesion information of a plurality of differential diagnosis frames in a case where the observation distance is the second observation distance and the diagnostic purpose is the differential diagnosis.

9. The processor device according to claim 8,
wherein, in the second display format for display, a lesion range is determined based on the lesion information of the plurality of range diagnosis frames, and the lesion information is displayed using the lesion range.

10. The processor device according to claim 8,
wherein, in the third display format for display, a differential content is determined based on the lesion information of the plurality of differential diagnosis frames, and the lesion information is displayed using the differential content.

11. The processor device according to claim 1,
wherein a display image for displaying the lesion information is obtained based on emission of first illumination light, and a lesion information acquisition image for acquiring the lesion information is obtained based on emission of second illumination light having a different emission spectrum from the first illumination light.

12. A method of operating a processor device including a processor, the method comprising:
the processor executing steps of:
acquiring an observation condition including at least one of a moving speed of an endoscope, an observation distance between the endoscope and an observation target, or brightness of the observation target;
acquiring lesion information including at least one of a diagnostic purpose or a certainty degree of a lesion obtained from an endoscope image at a timing at which the observation condition is acquired;
determining a display format of the lesion information on a display based on at least any of the observation condition or the lesion information; and
performing a control of displaying the lesion information on the display according to the display format.
